(19) United States
(12) Patent Application Publication
(10) Pub. No.: US 2012/0123235 A1
(43) Pub. Date: May 17, 2012

(54) IMPLANTABLE THERANOSTIC ARTICLE

(76) Inventors: Alexander BORCK, Aurachtal (DE); Andreas BUNGE, Leipzig (DE); Sarah BIELA, Berlin (DE)

(21) Appl. No.: 13/281,472

(22) Filed: Oct. 26, 2011

Related U.S. Application Data
(60) Provisional application No. 61/412,802, filed on Nov. 12, 2010.

(51) Int. Cl. A61B 5/00; A61M 5/00; A61N 1/36

(52) U.S. Cl. 600/365; 600/309; 607/2; 604/66; 604/93.01

(57) ABSTRACT

A theranostic article has one or more specific molecular recognition markers for cells on the surface thereof, wherein the recognition markers are selected from the group consisting of peptides, proteins, antibodies, antigens, aptamers, molecular imprinted polymers and polynucleotides. When the article is implanted in a body, cellular ingrowth is controlled, with desired cell types anchoring and proliferating on the implant's surface to generate a thin layer, and thereafter ceasing accumulation. The cellular layer thereby presents a biomimetic surface acceptable to the body, and also presents a low barrier to diffusion of analytes with at least substantially constant diffusion characteristics, allowing use of an analyte sensor within the article.

### CROSS-REFERENCE TO RELATED APPLICATIONS

This application claims priority under 35 USC §119(e) to U.S. Provisional Patent Application 61/412,802 filed Nov. 12, 2010, the entirety of which is incorporated by reference herein.

### FIELD OF THE INVENTION

The invention relates to an implantable theranostic article having specific detection markers for cells on its surface. The invention also relates to the use of specific recognition markers for cells for preventing biofouling and thrombogenicity on the surface of an implant. In addition, the invention relates to a method for producing an implantable theranostic article having a surface which reduces or eliminates biofouling.

### BACKGROUND OF THE INVENTION

A theranostic article (here also referred to as simply an “article”) has at least one diagnostic, sensor and/or therapeutic function. Such an article may also be designed as a theranostic implant (here also simply referred to as an “implant”).

Implants have great difficulty with long-term stability, especially when they are in contact with blood. After being introduced in the body, they typically exhibit non-specific protein adsorption. These adsorbed proteins at least partially lose their tertiary or quaternary structures and serve as anchor substrates for the deposition of cells. This triggers non-defined cell coverage and/or an extracellular matrix composed of protein fibers (such as collagen) on the surface of the article. This process is generally referred to as biofouling. In addition, it can lead to the formation of thrombi on the surface of the implants.

If the article is a sensor, these deposits can create a diffusion barrier for the analytes to be sensed, wherein the barrier is not stable over time and has a direct impact on the measurement result. The cell coverage and/or the extracellular matrix can result in drift, and also in a delay of the measurement signal, so that the sensor is able to detect exterior changes of the analyte concentration only with a time delay. The diffusion barrier may grow so large that the analytes can no longer reach the sensor, thereby preventing signal generation.

A variety of approaches have been described in the prior literature for preventing the aforementioned processes caused by the non-specific deposition of biomolecules, such as biofouling and the formation of thrombi, by modifying the surface of the article, i.e., by use of anti-biofouling coatings. Additionally or alternatively, an attempt is made to have the article's coating positively influence the response of the body wherein the article is implanted.

Frequently hydrophilic polymer structures, such as polyethylene glycol, are covalently attached to the surface of the article (PEGylation) in order to prevent the non-specific is protein deposits.

As an alternative, attempts are made to influence the ingrowth behavior of the introduced articles by micro- and nano-structuring the surface. For example, Gilligan et al. in “Feasibility of Continuous Long-Term Glucose Monitoring from a Subcutaneous Glucose Sensor in Humans”, Diabetes Technology & Therapeutics, Volume 6, Number 3, 2004, discloses an implantable sensor having a surface which is micro- and nano-structured to bring about positive ingrowth behavior. A comparable approach can also be found in Updike et al., “A Subcutaneous Glucose Sensor With Improved Longevity, Dynamic Range, and Stability of Calibration”, Diabetes Care, Volume 23, Number 2, February 2000.

In general, the articles are to remain in the body for an extended period of time. The body, however, reacts to the introduction of the article as a foreign object. In this connection, it is important that the responses of the body, such as inflammation or ingrowth, neither thwart the benefit of the article by causing stress to the body which exceeds the article's benefit, nor significantly impair the function of the article. For example, implantable sensors are medically only useful if they can remain in the body for an extended period of time, preferably at least one year, and supply stable measurement results for this duration. Up until now, no such implantable, long-term stable sensor systems are available, which supply the measurement results of sufficient quality for a year or longer. This is due in particular to biofouling. As discussed above, non-specific protein or cell deposits and thrombi from the body fluids can limit or even prevent the access for analytes to the actual sensor unit and thereby render the sensor useless. The temporal change in the analyte's access to the sensor region due to the gradual deposition and/or growth processes at the sensor surface also causes insufficient signal quality.

Owing to such factors, a semi-implantable system available from Medtronic (MiniMed Paradigm REAL-Time Revel™), used for the subcutaneous determination of glucose concentration, is (as of 2010) only approved by the US Food and Drug Administration (FDA) for use of no more than 7 days. In order to compensate for the signal drift by the aforementioned effects and additional error sources, calibrations by way of blood withdrawal are required several times a day.

Also as of 2010, no implantable or semi-implantable sensor is available in the market for molecular blood constituents other than glucose. An entire series of blood constituents, such as electrolytes, metabolites, bicarbonate, creatinine, urea, cystatin C, and other proteins would be useful for monitoring chronic diseases. At present, tests for such constituents are generally performed by drawing blood samples at the physician's office.

Surface modifications for reducing or suppressing biofouling by applying polymer coatings (for example, PEGylation) generally work sufficiently only for a short time after implantation of the article. When they contact body fluids, the polymers are relatively quickly chemically modified, which can result in a loss of the anti-biofouling properties. The surfaces provided with anti-biofouling properties additionally have the risk of triggering the coagulation cascade and therefore having a thrombogenic effect. It may therefore be necessary for an individual carrying a corresponding implant to be given long-term treatment with coagulation-inhibiting agents, such as dual antiplatelet therapy.

The use of nano- and micro-structuring for influencing tissue response to implants is relatively non-specific. While this approach attempts to present the tissue and the cells with topologies on the article's surface which can positively influence growth, it cannot control which cells accumulate on the surface. However, this is of great importance, especially when the implant is in contact with the blood flow. Prior analyses that have been conducted with implantable biochemical sensors have so far dealt only with subcutaneous use, while use of such sensors with nano- and micro-structured surfaces in sustained contact with blood flow has not thus far been described.

The two publications cited above can be listed as examples where structured surfaces are employed to subcutaneously influence the ingrowth behavior. The sensors described in these two publications are provided in each case with a membrane having a structuring, and prompting the tissue to form new blood vessels (angiogenesis). In this way, a fibrous encapsulation can be at least partially prevented. As a result, it was possible to extend the is service life, which is to say the duration over which the sensor supplies clinically usable measurement values lasted up to 5 to 6 months. However, it is not clear so far whether the described modifications of the surface can likewise positively influence biofouling, and whether use in lasting contact with blood is possible.

### SUMMARY OF THE INVENTION

Against the foregoing background, the present invention seeks to provide implants having a theranostic function, and which remain stable and functional in the body as long as possible, and having reduced (or preferably no) biofouling. These advantages are preferably exhibited upon contact with blood flow.

This objective can be achieved by an implantable theranostic article having specific recognition markers for cells on the surface thereof, wherein the recognition markers are selected from the group consisting of peptides, proteins, antibodies, antigens, aptamers, molecular imprinted polymers and polynucleotides having n≧1 monomer units (such as ribonucleic acid—RNA, deoxyribonucleic acid—DNA, peptide nucleic acid—PNA, locked nucleic acids—LNA).

Here, “molecular imprinted” refers to the provision of a polymer backbone with recognition domains. Molecular imprinting provides access to polymers carrying information. For example, polymers can be provided with the necessary selectivity to form affinities for similar structures by radical polymerization in the presence of a template (which in the most favorable case can be removed again by employing washing steps). Such a method is known from Vaidya, A., Borck, A., Manns, A. & L. Fischer. 2004: “Altering glucose oxidase to oxidase Dgalactose through crosslinking of imprinted protein” (ChemBioChem 5 (1): 132-135).

The type of the epitope determines the polymer selection for the molecular imprinted polymer(s). Acrylates and methacrylates are suitable as the skeleton. AIBN is the preferred radical starter. The reaction can be carried out in solvents such as dioxane, CHCl3 or THF, or also in substance. In addition to the high selectivity, high binding affinities can be is produced by co-polymers with acylacetate (hydrophilic groups; can be saponified to produce OH groups), acylamine, acylic acids or styrol (interaction with aromatic epitopes).

A “specific recognition marker” refers to a molecule that is specifically designed for the deposition of certain cell types. In other words, a “recognition marker for cells” refers to a compound, or part of a compound, which is specifically recognized by one or a few cell types, preferably one or otherwise less than four, and can bring about binding of the cells of this type or of these types to a surface on which the recognition marker is located. Cells of other types, in contrast, do not show such a reaction. In a preferred version, the migration and proliferation of endothelial cells (EC) is promoted.

In general, the preferred cell types are those which recognize the recognition markers on the implant surface with the help of transmembrane proteins (integrins), selected from the group of the cells that carry integrins. Cells that are part of the alphaBeta3 (avβ3) subfamily are particularly preferred. In addition to the endothelial cells already mentioned above, endothelial precursor cells also contain the desired recognition sequences. See, e.g., Blindt et al.: “A novel Drug-Eluting Stent coated with an Integrin-Binding Cyclic Arg-Gly-Asp Peptide Inhibits Neointimal Hyperplasia by Recruiting Endothelial Progenitor Cells”; JA College of Cardiology; Vol. 47, No9, 2006; also Garcia A. J.: Get a Grip, integrins in cell-biomaterial interactions”; Biomaterials 26; 7525-7529, 2005.

The use of peptide sequences on stents is described in WO 2008/143933 A1. Here, accelerated healing through the formation of a cell layer is also to be achieved.

The described method is not known yet for theranostic applications. Efforts to control and/or prevent tissue coverage, such as by PEGylation, are known and are the state of the art.

With a suitable selection of the markers and depending, for example, on the site at which the implantable theranostic article is to be used, the provision of certain recognition markers on the surface of implantable theranostic articles causes the implanted article's surface to present an interface to which the body reacts with a defined thin scarring or encapsulation that does not change further after a certain period of time. In this respect, it is particularly surprising that following a relatively short growth attachment phase, tissue develops on the implanted theranostic articles which practically does not change any further. Given this biomimetic surface, the body no longer identifies the article as a foreign object.

The invention therefore represents a new approach. While past attempts primarily focused on creating bioinert surfaces and nano-structurings for implant applications, which were associated with the disadvantages of thrombogenicity and lacking specificity, the present invention achieves the creation of a (largely) constant and biomimetic surface through the use of specific recognition markers for cells. To this end, the scope of the cell coverage can also be controlled by the concentration of the specific recognition markers for cells on the surface of the implantable theranostic article. In principle, however, relatively low cell coverage is created, which after a growth attachment phase (or healing) does not increase further over time. As a result, this layer constitutes a surprisingly low diffusion barrier, and it is likewise surprising that the ability of analytes to diffuse through the barrier is at least substantially constant.

It is preferred for the recognition markers to be bound to the implantable article by way of adsorption, a covalent bond, or a linker. Using a suitable bonding method, a person skilled in the art will be able to control the effective ingrowth behavior of the (desired) cell type.

Article surfaces that are suitable for absorption are preferably selected from the group consisting of titanium, medical stainless steel such as preferably 316L, CoCr, magnesium, and polymers. Under the usage conditions, polymers may be both decomposable in the body or be permanent on the body.

Groups that are suitable for covalent bonds on the surface of the implantable article are preferably selected from the group consisting of hydroxy radical, amino radical, carbonyl radical and mercapto group.

A linker within the meaning of the present invention is a molecule part that chemically ensures the connection between the specific recognition marker for cells and the surface of the implantable theranostic article. The linker comprises an anchor group and a spacer group. The spacer group has a chain length of 1-30 atoms, with 5-12 atoms being preferred. Suitable preferred anchor groups include: acylic acid, phosphonates, thiols, and isocyanates, with isothiocyanates being particularly preferred. Preferred spacers include: PEG, polyproline, and adipic acid, with aminohexanoic acid being preferred.

Under certain usage conditions, certain reagents for coupling N,N′-carbonyldiimidazole (CDI), 1-(3-dimethylaminopropyl)-3-ethylcarbodiimide (EDC) or disulfosuccinimidyl-tatrate (DST) are likewise preferred.

A preferred specific recognition marker is an oligopeptide. Oligopeptides comprise up to ten amino acids and, given the size thereof and the functional principle thereof, can be employed particularly well as recognition markers for certain cell types.

A particularly preferred implantable article utilizes a molecular recognition marker including an RGD or cRGD sequence, or is made thereof.

It is particularly preferred for the specific recognition marker for cells to be selected from the group consisting of compounds of formula I:

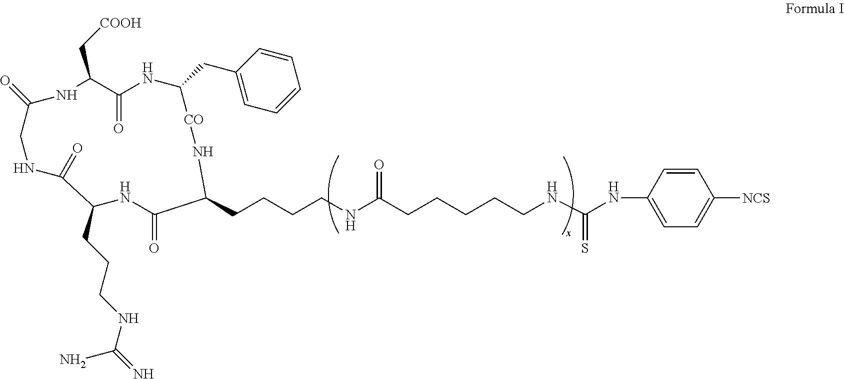

x=0, 1, 2, 3, 4, 5 or 6
and
compounds of formula II

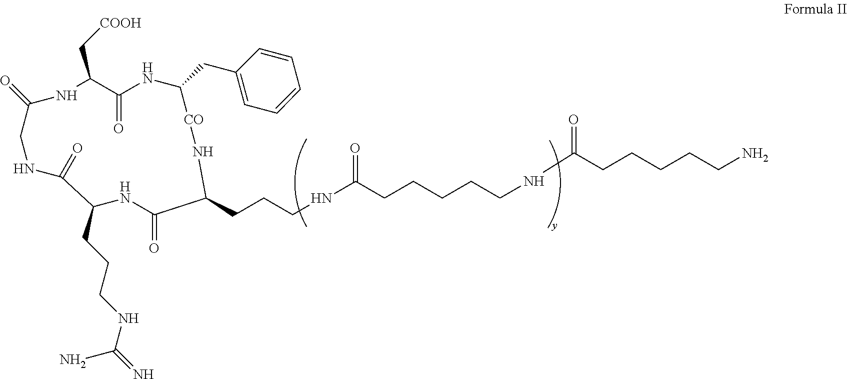

y=0, 1, 2, 3, 4 or 5.

It is further preferred for the surface of the article to be of a metallic, ceramic or polymer nature.

In addition to including the specific recognition markers for cells, the surface of the implantable theranostic article can be passivated against the deposition of interfering factors from the body, particularly from body fluid (provided the implantable article comes in contact with body fluid). This can be done, for example, by way of suitable polymers or hydrogels, for example on the basis of polyethylene glycol (PEG).

The surface of the implantable theranostic article can bear further modifications that bind or reject specific constituents of the body fluid. Such modifications can be inorganic or organic molecules that are tied to the article surface by way of physical absorption or a covalent bond, such as polymers, peptides, proteins, aptamers, molecular imprinted polymers, RNA, DNA, PNA, LNA, siRNA, and nano-particles.

The surface of the articles according to the invention can bear nano- or micro-structuring. In order to structure the surface, structures having desired shapes may be applied or removed, e.g., round, spherical, cylindrical, conical, square, rectangular or elongated structures, including grooves, tubes, full cylinders, spheres, semi-spheres, cuboids and cubes.

The implantable theranostic article preferably includes an active substance dosing system. An active substance dosing system here refers to an implantable article that can dispense a drug in a controlled manner over an extended period of time. Closed-loop systems according to the invention combine a sensor system measuring certain parameters in body fluids, such as glucose, with an active substance dosing system, for example for dispensing insulin, so that critical physiological values can be corrected quickly, or the occurrence of certain physiological events can be prevented. Surface modification is particularly well-suited for this type of implantable theranostic article, because many of these articles typically come in contact with body fluid, in particular blood, where defined ingrowth has particularly high significance.

In such a sensor system, it is (as noted above) particularly surprising that a sensor having a surface coverage with specific recognition markers for cells can be achieved for the implantable theranostic articles, wherein the resulting cell layer is sufficiently permeable for the respective analytes. This even applies if the part of the surface of the implantable sensor that is formed by a semipermeable membrane (permeable to the desired analyte) also bears specific recognition markers for cells on its surface.

The theranostic article preferably defines a sensor as described above, wherein the sensor is a sensor for a molecular constituent of a body fluid. Here “molecular constituent” refers to a constituent of the body that is formed directly by the body and/or received by it, such as an electrolyte, and a molecule of the group consisting of carbohydrates, metabolites, peptides, fats, lipids, proteins, neurotransmitters, polyelectrolytes, nucleotides, hormones, nanoparticles, or active substances, amino acids, fatty acids, deoxyribonucleic acid, ribonucleic acid, and water. A preferred article includes a sensor for a blood constituent.

A glucose sensor is particularly preferred. Glucose is a relevant analyte owing to diabetes diseases, wherein real-time control of the blood glucose level is desirable. Diabetes mellitus is a widely common disease with several million affected patients in the USA alone. At the same time, glucose is particularly suited as an analyte because it is a relatively small molecule and thereby ensures good diffusion behavior.

Also preferred are analytes selected from the group consisting of electrolytes, carbohydrates, metabolites, amino acids, peptides, fats, fatty acids, lipids, proteins, neurotransmitters, polyelectrolytes, ribonucleic acid, deoxyribonucleic acid, nucleotides, hormones, nanoparticles, active substances, and water. Particularly preferred are: albumins/globulins, alkaline phosphatase, alpha-1-globulin, alpha-2-globulin, alpha-1-antitrypsin, alpha-1-fetoprotein, alpha-amylase, alpha-hydroxybutyrate-dehydrogenase, ammonia, antithrombin III, bicarbonate, bilirubin, carbohydrate antigen 19-9, carcinoembryonic antigen, chloride, cholesterol, cholinesterase, cobalamin/Vitamin B12, coeruloplasmin, C-reactive protein, cystatin C, d-dimers, iron, erythropoetin, erythrocytes, ferritin, fetuin A, fibrinogen, folic acid/Vitamin B9, free tetraiodothyronine (fT4), free triiodothyronine (fT3), gamma-glutamyltransferase, glucose, glutamate dehydrogenase, glutamate oxalacetate transaminase, glutamate pyruvate transaminase, glycohemoglobin, packed cell volume, hemoglobin, haptoglobin, uric acid, urea, HDL cholesterol, homocysteine, immunoglobulin A, immunoglobulin E, immunoglobulin G, immunoglobulin M, INR, potassium, calcium, creatinine, creatine kinase, copper, lactate, lactate dehydrogenase, LDL cholesterol, leukocyte, lipase, lipoprotein, magnesium, mean corpuscular hemoglobin concentration, mean corpuscular hemoglobin, mean corpuscular volume, myoglobin, sodium, NT-proBNP/BNP, osmolality, partial thromboplastin time, phosphate, pH value, plasma thrombin time, prostate-specific antigen, prothrombin time, reticulocytes, rheumatoid factor, thrombocytes, thyreoidea stimulating hormone, transferrin, triglycerides, troponin T.

The term “active substance” includes typical pharmaceuticals, or metabolites, which are issued for treating diseases and are of interest as active substances, for example muscarinic receptor antagonists, neuromuscular blocking agents, cholesterol esterase inhibitors, adrenoceptor agonists, indirectly acting sympathomimetic drugs, methylxanthine, alpha-adrenoceptor antagonists, ergot alkaloids, beta-adrenoceptor antagonists, inactivator inhibitors, antisympathonic drugs, 5-HT receptor agonists, histamine receptor agonists, histamine receptor antagonists, analgesics, local anesthetics, sedatives, anticonvulsant drugs, convulsant drugs, muscle relaxers, anti-Parkinson's drugs, neuroleptics, antidepressants, lithium, tranquilizers, immunosuppressants, anti-rheumatism drugs, antiarrhythmic drugs, antibiotics, ACE inhibitors, aldosterone receptor antagonists, diuretics, vasodilatators, positive inotropic agents, antithrombotic/thrombolytic substances, laxatives, antidiarrheal drugs, pharmaceuticals for adiposity, uricostatic drugs, uricosuric drugs, lipid lowering drugs, antidiabetics, antihypoglycemic drugs, hormones, iodized salts, threostatic drugs, iron, vitamins, trace elements, virostatic, antimycotics, antitubercular drugs, and substances for tumor chemotherapy.

Typical pharmaceuticals or metabolites that are of interest include those administered for coronary heart disease and cardiac insufficiency, such as diuretics, ACE inhibitors (Ramipril, Captopril), beta-blockers (Carvedilol), angiotensin receptor blockers (Valsartan), aldosterone blockers (Eplerenone, Spironolacton), and statins (Atorvastatin).

Many of these analytes can be determined in body fluids to allow characterization of the physical conditions of individuals, particularly in the case of chronic diseases such as cardiac insufficiency or renal insufficiency. The majority of molecules of interest are small enough to ensure good diffusion behavior through an endothelial cell layer into the interior of the sensor.

The implantable theranostic article preferably also includes a telemetry unit (transceiver) which allows the determined values of the analyte concentration to be transmitted unidirectionally or bidirectionally to an external device, which may display the determined values. It is also possible for the implantable theranostic article to contain an active substance dosing system, which automatically dispenses an active substance, for example insulin, depending on the analyte concentration that is determined (closed loop). It is furthermore possible for data to be transmitted from the external device to the active substance dosing system, serving as a trigger for the dispensing of the substance. This data is transmission can take place automatically. As an alternative, it is possible for a trigger for the active substance dosing system to be set manually.

Implantable articles according to the invention should function for at least three months after implantation, preferably at least six months, with at least one year being particularly preferred.

The invention also relates to the use of specific recognition markers for cells, wherein the recognition markers are selected from the group consisting of antigens, peptides, proteins, antibodies, aptamers, molecular imprinted polymers and polynucleotides having n≧1 monomer units (DNA, RNA, PNA, LNA), for preventing biofouling and/or the formation of thrombi on the surface of an implant. Such a use results in the advantages described above, wherein the ingrowth behavior of the implants in the implanted state is controlled in a defined manner. The recognition markers are bonded to the surface of the implant to act as attraction points for certain (desired) cell types, which are immobilized on the implant's surface and thereafter undergo proliferation, thereby creating homogeneous (thin) cell coverage of the implant. As also discussed above, this presents the body with a natural surface so that defense reactions of the body, such as inflammatory reactions, are mitigated or suppressed, as is the formation of thrombi. After the ingrowth phase, the minimal cell coverage reaches a constant state, i.e., it does not increase further over time, and provides only a low (and in particular constant) diffusion barrier. Accordingly, if the theranostic implant is a sensor, an analyte can reach the inside of the sensor relatively easily, and with a uniform analyte concentration, the signals of the sensor remain constant, and therefore reproducible, for a long period of time. Due to the stationary and unchanging state of the implant surface, high long-term stability of the implant is possible.

The invention further relates to a method for producing an implantable theranostic article having a biofouling-reducing or biofouling-preventing surface and/or a surface for reducing or preventing the formation of thrombi, wherein the production method includes the following steps:

a) providing an implantable article, and
b) providing the surface of the article, or parts thereof, with specific recognition markers for cells, wherein the recognition markers are selected from the group consisting of antigens, peptides, antibodies, aptamers, molecular imprinted polymers and polynucleotides having n≧1 monomer unit (RNA, DNA, PNA, LNA).

A person skilled in the art can employ suitable further steps to produce the various preferred and other versions of the implantable articles described above. The advantage of such articles is that they may be employed in many areas of the body, that is, not only subcutaneously for example, but also intravasally or otherwise in contact with blood flow. The skilled artisan can suitably select the specific recognition markers desired, and suitably apply these recognition markers on the article surface. Since the selection of the markers defines the cell type that is preferably deposited on the surface of the article, the ingrowth behavior can be controlled. The skilled artisan will of course select the specific cell recognition marker depending on the desired site of use, and depending on the intended use of the implant, such as in a human or in a certain animal.

### DESCRIPTION OF THE DRAWING

FIG. 1 shows a schematic illustration of an exemplary implantable theranostic article in accordance with the invention.

FIG. 2 shows a schematic illustration of the molecular imprinting of polymers.

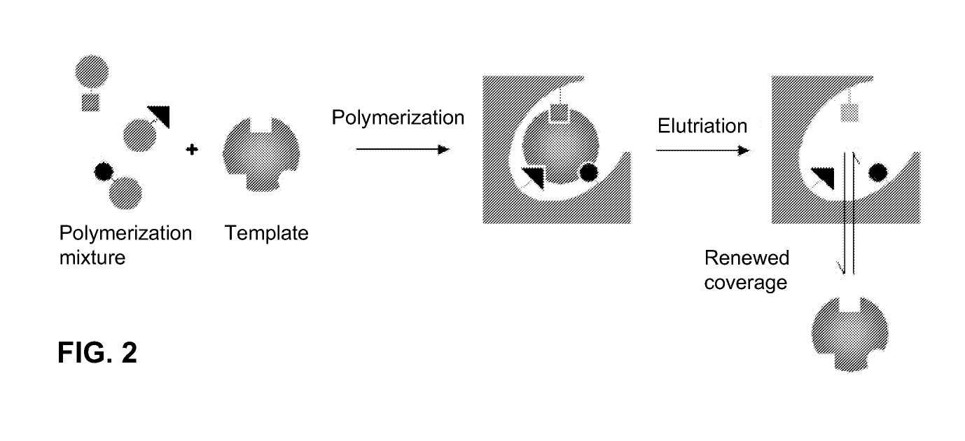

### DETAILED DESCRIPTION OF EXEMPLARY VERSIONS OF THE INVENTION

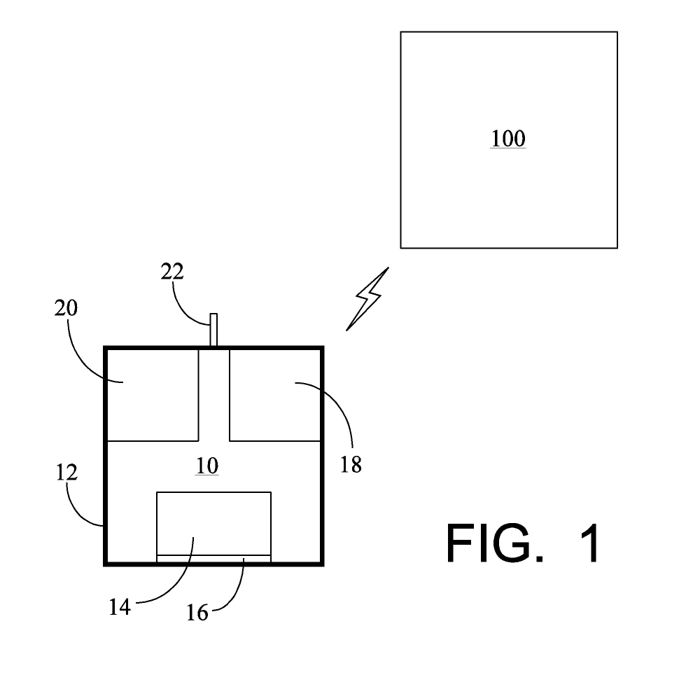

An exemplary version of the implantable theranostic article is illustrated in the accompanying FIG. 1. The theranostic article 10, which is implanted within a human or animal body (not shown), is coated on its outer surface 12 with a molecular recognition marker configured to bind one or more specific types of cells. The article 10 includes a sensor 14, e.g., a glucose sensor, having a semipermeable membrane 16 defined at the is outer surface 12 of the article 10. This sensor 14 may be of the type noted in U.S. patent application Ser. No. 13/253,121 filed Oct. 5, 2011 (the contents of which are incorporated by reference herein). The article 10 additionally includes a telemetry unit 18 configured to transmit a concentration of the constituent detected by the sensor 14 to an external device 100, and an active substance metering system 20 configured to dispense an active substance from the article 10, e.g., in dependence on a signal from the sensor 14 and/or from the external device 100.

While the article 10 is depicted as a glucose-sensing active substance dosing system, the principles described herein can be applied to other theranostic articles, for example, active implants such as cardiac pacemakers, defibrillators, cardioverters or neurostimulators. The article 10 may therefore include features such as an electrode 22 (e.g., an electrode-bearing electrode line) configured to deliver electrical stimulation to the body wherein the article 10 is implanted.

The glucose sensor in the following examples is based on the principle of traditional amperometric enzyme sensors with immobilized glucose oxidase. Here, the glucose is measured selectively by the enzymatic conversion of glucose. The glucose oxidase enzyme is immobilized in the sensor tip using a polymer and cross-linked with glutaraldehyde. What is measured is either the decrease in oxygen or the formation of hydrogen peroxide using an electrochemical reaction of the glucose with the oxidase. Either oxidation on the electrode occurs, or a reduction at the counter-electrode.

The articles coated in examples 1-3 below are implanted in a large, peripheral vein. Initially, they are not to be parietal, but fastened freely in the blood flow. The special coating attracts endothelial progenitor cells from the blood flow and endothelial cells, which settle on the article surface, proliferate, and form a monolayer endothelium after a few days. The endothelial cells also grow over the semi-permeable sensor window, but form sufficiently large inter-cell pores for the analyte to diffuse through the window.

### Example 1 of a Surface Coating:

A sensor head, which has been cleaned in oxygen plasma or by spraying with a series of solutions of dichloromethane, acetone, methanol and Millipore water, is treated further as follows:

A 1 mM solution of hydroxyundecyl phosphonic acid in dry tetrahydrofurane (THF) is produced. The sensor head is hung in this solution, and the solvent is evaporated within one hour, whereby the meniscus of the solution migrates over the sensor surface.

Thereafter, the temperature of the sensor head is controlled for 18 hours at 120° C., and the head is then rinsed with the solvent THF.

The surface pretreated in this manner is placed for 15 hours in a 0.3 M solution of carbonyldiimidazole (CDI) in dry dioxane. Afterwards, the substrate is rinsed twice for 10 minutes with dry dioxane and then dried in a flow of nitrogen.

A solution of the compounds to be bound (here, a cyclic pentapeptide according to the foregoing formula II, where y=2 (approx. 50 μg/ml) in PBS buffer (amino acid free) is placed on the surface treated in this way and shaken over night at 4° C. Then, the sensor head is rinsed with buffer.

### Example 2 of a Surface Coating:

A sensor housing made of titanium (Ti), which has been cleaned in accordance with Example 1 and is made of a cylinder having a diameter of 3-7 French, having a lead-through for a sensor cable at one end of the head, and a sensor window composed of a semi-permeable membrane at the other end, is treated further as follows:

A 3 mM solution of 3-(4-oxybenzophenone)propyl phosphonic acid in dry tetrahydrofurane is produced.

This solution is sprayed three times on the cleaned surface. Thereafter, the temperature of the housing is controlled for 12 hours at 120° C., and the housing is then rinsed with the solvent THF.

The titanium housing is placed in a solution of the compounds to be bound (here, a cyclic pentapeptide according to the foregoing formula II, where y=2 (approx. 50 μg/ml) in PBS buffer according to Example 1 and shaken over night at 4° C.

The next day, the Ti sensor surfaces are removed from the solvent, dried, and exposed at 260 nm with 100 mW/cm².

Protein that is not bound is washed off.

### Example 3 of a Surface Coating:

The cleaned sensor housing made of titanium (see Example 2) is placed in a mixture of toluene, triethylamine and 3-aminopropyltriethoxy silane and incubated for 14 hours at room temperature. After the reaction is complete, the sensor is washed in toluene and the temperature is controlled for 1 hour at 135° C.

### Composition of the Silanization Solution:

10 ml toluene, dried
0.5 ml trietylamine
1 ml silane 3-aminopropyltriethoxy silane

The cleaning step (rinsing the Ti substrate with trichloromethane) is followed by the activation with 1,1′-carbonyldiimidazole (CDI).

The silanized and rinsed Ti substrates are placed in CDI for 5 hours. For this purpose, the CDI is dissolved in dry dioxane. A parent solution of 2.5 g/50 ml CDI in dioxane is suited for this, which holds for several days (2, dry). The substrates are moved slightly at room temperature.

After the activation, the substrates are removed and rinsed with dry dioxane.

For binding the cyclic peptides according to the foregoing Formula I with x=2, the activated Ti substrates are immersed in the peptide solution having a concentration of 5 mg/ml and bound at 4° C. over night (at least 12 hours).

The reaction is suitably carried out in 125 mM sodium borate with 0.066% SDS at a pH value of 10.0.

The solution can then be reused, and/or several surfaces can be treated using this solution.

After binding, the sensors are washed three times with 5 ml of the Borax buffer (above). Then they are rinsed another three times with water. The peptides that can still be analyzed after these washing steps are covalently bound.

All of the implantable sensors coated in Examples 1 to 3 exhibited defined, single-layer, temporally unchanged and non-thrombogenic scarring after implantation and the ingrowth phase. The sensors were all operational for at least 6 months. The semi-permeable membranes of the sensors remain constantly permeable to the analytes, so that reliable and reproducible signals were generated.

It will be apparent to those skilled in the art that numerous modifications and variations of the foregoing examples and versions are possible in light of the discussion above. The foregoing examples and versions are presented for purposes of illustration only, and the invention encompasses all versions, variations and alternatives that are described by the claims below, or are equivalent to such versions, variations and alternatives.

What is claimed is:

1. An implantable theranostic article having a surface bearing a molecular recognition marker on at least a portion of the surface thereof, the recognition marker being configured to bind one or more specific types of cells, wherein the recognition marker is selected from the group consisting of peptides, proteins, antibodies, antigens, aptamers, molecular imprinted polymers and polynucleotides.

2. The implantable theranostic article of claim 1 wherein the recognition marker is bound to the surface of the article by adsorption, a covalent bond, or a linker.

3. The implantable theranostic article of claim 1 wherein the recognition marker is bound to the surface of the article by a linker, the linker including:
a. an anchor group including one or more of isothiocyanates, isocyanates, acylic acid, phosphonates, and thiols, and
b. a spacer group including aminohexanoic acid, polyethylene glycol, polyproline, and adipic acid.

4. The implantable theranostic article of claim 1 wherein the recognition marker is an oligopeptide.

5. The implantable theranostic article of claim 1 wherein the recognition marker includes an RGD or cRGD sequence.

6. The implantable theranostic article of claim 1 wherein the recognition marker is configured to bind endothelial cells.

7. The implantable theranostic article of claim 1 wherein the recognition marker is configured to bind alphabeta3 cells.

8. The implantable theranostic article of claim 1 wherein the recognition marker includes one or more of:
a. the following compound wherein x is greater than or equal to zero: 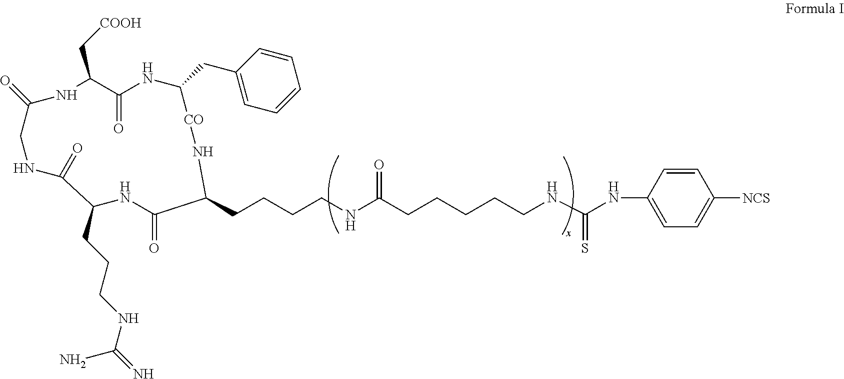
x=0, 1, 2, 3, 4, 5 or 6
b. the following compound wherein x is greater than or equal to zero: 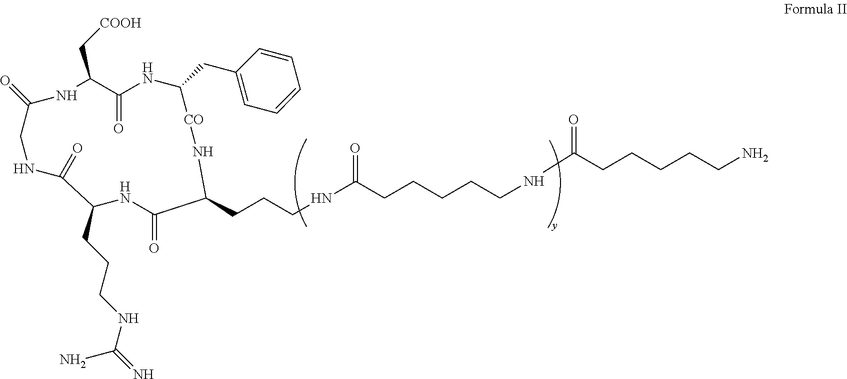
y=0, 1, 2, 3, 4 or 5.

9. The implantable theranostic article of claim 1 wherein the article includes an active substance metering system configured to dispense an active substance from the article.

10. The implantable theranostic article of claim 1 wherein:
a. the article includes a semipermeable membrane, and
b. at least a portion of the membrane bears the recognition marker thereon.

11. The implantable article of claim 1 wherein the article includes a sensor configured to detect a molecular constituent of a body fluid.

12. The implantable theranostic article of claim 11 wherein the sensor is configured to detect a molecular constituent of blood.

13. The implantable theranostic article of claim 11 wherein the sensor is configured to detect glucose.

14. The implantable theranostic article of claim 11 wherein:
a. the sensor bears a semipermeable membrane, and
b. at least a portion of the membrane bears the recognition marker thereon.

15. The implantable theranostic article of claim 11 further including an active substance metering system configured to dispense an active substance in dependence on a concentration of the constituent detected by the sensor.

16. The implantable theranostic article of claim 11 further including a telemetry unit configured to transmit a concentration of the constituent detected by the sensor to an external device.

17. The implantable theranostic article of claim 16 in combination with an external device configured to:
a. receive the transmitted concentration of the constituent, and
b. transmit a trigger signal from the external device to the article.

18. The implantable theranostic article of claim 1 wherein the article is defined by an active implant configured to deliver electrical stimulation to a body wherein the article is implanted.

19. A method for producing an implantable theranostic article including the steps of:
a. providing an implantable article, and
b. providing at least a portion of the surface of the article with a molecular recognition marker configured to bind one or more specific types of cells, wherein the recognition marker is selected from the group consisting of peptides, proteins, antibodies, antigens, aptamers, molecular imprinted polymers and polynucleotides.

20. A method for at least partially preventing biofouling and/or the formation of thrombi on the surface of an implantable theranostic article, the method including the step of providing at least a portion of the surface of the article with a molecular recognition marker configured to bind one or more specific types of cells, wherein the recognition marker is selected from the group consisting of peptides, proteins, antibodies, antigens, aptamers, molecular imprinted polymers and polynucleotides.
